(19) United States
(12) Patent Application Publication
(10) Pub. No.: US 2005/0048671 A1
(43) Pub. Date: Mar. 3, 2005

(54) Gap junction-containing devices for detecting biochemical activity

(75) Inventors: Christoph Methfessel, Wuppertal (DE); John Goossens, Leverkusen (DE)

Correspondence Address: NORRIS, MCLAUGHLIN & MARCUS, PA, 875 THIRD STREET, 18TH FLOOR, NEW YORK, NY, 10022, US

(73) Assignee: Bayer Technology Services GmbH, Leverkusen (DE)

(21) Appl. No.: 10/912,385

(22) Filed: Aug. 5, 2004

(30) Foreign Application Priority Data
Aug. 26, 2003 (DE) 10339597.0

(51) Int. Cl. G01N 33/558

(52) U.S. Cl. 436/514

(57) ABSTRACT

A device for spatially separating a biochemical activity from a detection reaction that detects the biochemical activity. The device comprises a first membrane body comprising the biochemical activity, and a second membrane body comprising the detection reaction. The first and second membrane bodies each comprise lipid bilayers, wherein the first and second membrane bodies are connected by gap junctions.

The invention relates to devices and methods for detecting biochemical activity using a detection reaction, with the biochemical activity to be detected proceeding in a manner in which it is spatially separated from the detection reaction. The spatial separation is achieved using lipid membranes and gap junctions.

### BACKGROUND

Connexins, Connexons and Gap Junctions

The skilled person is familiar with biological protein molecules, termed connexins, which play a special role in communication between living cells. By now, approximately fifteen different connexins have been distinguished on the basis of their amino acid sequences [1][2]. Connexins are found in all vertebrates and are as a rule designated by means of an abbreviation such as Cx26. The number in the abbreviation indicates the chromatographic size of the connexin in kD. The connexins which are known to date have molecular weights ranging between 26 and 56 kD. As an alternative to this nomenclature, a second has become accepted, with this latter dividing up the connexins into at least 3 classes a, b and c on the basis of structural features and then numbering the corresponding connexins consecutively in the individual classes.

In the cell membrane, six connexins in each case associate to form a connexon. A connexon is an annular structure which traverses the cell membrane and is in principle able to form a very wide, nonspecific ion channel or a water-filled pore. However, these pores are as a rule closed as long as the connexon is present in the membrane of a single healthy cell. However, if two cells which possess mutually compatible connexons in their membrane come into contact, a gap junction (otherwise termed an electric synapse) is then formed between two connexons in the opposing cells with this gap junction then bridging the two membranes and the distance between the cell membranes. As a rule, when contact occurs, a gap junction is formed in a few minutes. The gap junction channel which has been formed is a structure which as a rule consists of 12 identical or different connexins or two connexons. The channel possesses an optionally sealable central pore which has a diameter of from about 1.5 to 2 nm. The essential difference as compared with other membrane channels is that gap junction channels pass through two juxtaposed cell membranes, thereby establishing a connection between the intracellular media in the two cells rather than a connection between the interior of the cell and the external medium.

In this connection, gap junction channels enable inorganic ions and small water-soluble molecules having a molecular mass of up to approx. 1000 daltons to pass directly from the cytoplasm of the one cell into the cytoplasm of the other cell. In this way, the two cells are connected both mechanically and electrically and metabolically as well. Gap junction channels are epithelial cell-cell compounds and are found in virtually all epithelia and many other types of tissue. As a rule, a large number of gap junction channels are organized in the form of zones, with these structures then being termed gap junctions in the true sense.

As a rule, the gap junction channels belonging to the cells which are connected are open and the connexins are extended. However, if a cell experiences a massive influx of calcium from the exterior, for example as a result of an injury, the connection with neighbouring cells is interrupted due to the connexins intertwining with each other in an allosteric manner.

Connexins can be obtained by purifying them from the membranes of cells which contain connexins, for example eye lens, heart muscle, smooth musculature or epithelial cells, and also by means of using genetic manipulation to express the connexins in bacteria, yeasts or other cells. It is furthermore known that connexins can be provided, by combination, with a label, such as a fluorescent protein fragment, so as to enable their presence in a cell membrane to be detected using simple optical methods [3].

The skilled person is familiar with methods which can be used to incorporate connexons into artificial membranes or other cell-free systems [4]. These connexons and gap junctions frequently still exhibit the same properties, such as pore size, ion selectivity and electrical behaviour, as they do in their natural environment. It is known that an operable gap junction channel is also formed, when the membrane surfaces come into contact, between two connexons which are embedded in artificial membranes [5].

It is furthermore known that invertebrates possess a functionally similar class of membrane proteins which are termed innexins [6]. However, the channels which are formed from these proteins possess a larger pore which permits the passage of molecules having a weight of up to 2000 daltons. Innexins also form gap junctions within the meaning of the invention.

In addition, it is known that connections between cells also occur in plants, with these connections possessing properties which are similar to those of gap junctions and being termed plasmodesmata. These connections likewise span the partition wall between neighbouring cells and also enable a limited number of ions and small molecules to pass from cell to cell. However, in contrast to the channels in animal life-forms, the plasmodesmata are bounded by the plasma membrane. Within the meaning of the invention, these structures are also encompassed by the generic term gap junction.

Cellular Assays

Cellular assays are also known in which a particular biochemical activity, which take place within a living cell, are detected using suitable detection reactions which also take place within the cell. In particular, methods are known in which reaction products of the biochemical activity react, within the cell, with indicator molecules which then elicit a readily detectable signal, for example a light signal or a colour change. An example of an indicator molecule which is frequently employed is aequorin [7].

A feature possessed in common by these cellular assays is that they are restricted to the use of indicator molecules which do not have any toxic effect on the cells and which are able to perform their function within the living cell. Otherwise, the cells would be damaged by the toxic indicator molecules before or during the assay such that it would no longer in any way be possible to carry out the actual test.

Assays Performed on Artificial Lipid Membranes

It is also known that ion channels, receptors and other target molecules are incorporated into artificial lipid membranes and that suitable experiments can then be used to investigate their function in these membranes [8]. Of particular interest are the methods of stably supporting an artificial lipid membrane using a suitable substrate such that the membranes become mechanically stronger, more durable and more reproducible [9]. An example of substrates which are used in this connection are silica gels which have, where appropriate, been provided with a polymeric intermediate layer for the purpose of improving the stability and fluidity of the membrane [10]. It is also possible to use suitable macromolecules to stabilize bilayers (tethered bilayers) on the substrate [11].

The prior art contains methods in which target molecules (e.g. ion channels, receptors, enzymes, etc.) are introduced into a lipid bilayer which is immobilized on a spherical solid substrate. The biochemical activity of these target molecules can then be detected using indicator molecules which are located below the lipid bilayer [10]. The substrate, which is mainly solid, is, for example, a silica support to which a lecithin lipid bilayer has been applied. Such an arrangement can be attained commercially, inter alia, from Nimbus Biotechnologie (Leipzig, Germany) under the trade name Transil®. In this arrangement, calcium-sensitive or phosphate-sensitive dyes, which are immobilized below the lipid bilayer, are used to detect the biochemical activity of target molecules (which are embedded directly in the lipid bilayer).

However, the above-described method suffers from the disadvantage that substrates which are appropriately coated and which are enriched with the target molecule have to be prepared for investigating each and every target molecule. In addition, it is not always possible to ensure that, in this “artificial” environment, the target molecules will display the same biochemical activities as they do in the “natural” cellular environment.

### DESCRIPTION OF THE INVENTION

Based on the above-described prior art, the technical object which presents itself here is that of providing improved methods and devices in connection with which it is possible, on the one hand, to employ indicator systems (e.g. toxic detection reagents) which cannot be used for cellular assays and, on the other hand, it is possible to analyze the investigated target molecules in their natural cellular environment.

According to the invention, the technical object is achieved by using devices and methods in which the biochemical activity to be investigated takes place in a manner in which it is spatially separated from the reaction which detects the biochemical activity. This makes it possible, for example, to use toxic detection reagents for detecting the biochemical activity while the target molecule is still displaying the biochemical activity to be detected in its natural cellular environment. The biochemical activity and the detection reaction are spatially uncoupled.

According to the invention, the spatial separation is achieved by sites in which the biochemical activity to be investigated and, respectively, the detection reaction take place being separated by at least two lipid membranes. The lipid membranes are in each case provided with connexins or innexins such that gap junctions are formed between the membranes, with these gap junctions making it possible for molecules or other signals to pass between the site of the biochemical activity and the site of the detection reaction.

Within the meaning of the invention, “biochemical activity” is any biochemical activity which takes place, which can take place, or which can be catalyzed by the biological system, in or on, or at the surface of, biological systems (e.g. cells). Examples of biochemical activities are protein-catalyzed chemical reactions, signal transduction processes or changes in the physical or chemical state variables (such as pH, ion concentration, metabolite concentrations, etc.). The proteins which constitute target molecules can be present in the dissolved state, for example free in cytoplasm, or else be bound to membranes, e.g. to the cytoplasmic membrane or to organelles.

Within the meaning of the invention, “detection reactions” are chemical or biochemical reactions which can detect, or can render detectable, the biochemical activity or its consequences. Detection reactions which are preferred are colour reactions, fluorescent or luminescent phenomena or complex biochemical reactions which enable the biochemical activity to be detected.

Within the meaning of the invention, “lipid membranes” are lipid membranes as are known to the skilled person from biological or nonbiological systems. Lipid membranes preferably contain a lipid bilayer which prevents the free passage of hydrophilic substances. Lipid membranes within the meaning of the invention can have molecules, e.g. proteins, which are embedded in them. Lipid membranes may have a spherical or planar shape. They can also, in particular, be present on a solid or gelatinous substrate. One particular embodiment of the lipid membrane is a lipid membrane composed of lecithin.

Within the meaning of the invention, “gap junctions” are connections between two three-dimensional regions which are separated from each other by lipid membranes. Gap junctions are formed from proteins which span the lipid membranes and the space between the membranes and in this way create a passage for substances and for charge exchange.

Within the meaning of the invention, “membrane bodies” are volume elements which are enclosed by a membrane and which are filled with a liquid. Membrane bodies according to the invention are preferably biological membrane bodies, such as living cells. These living cells include cells which have been isolated from living tissue by means of dissociation (primary cultures). They also include cells which are maintained in culture as established cell lines, such as CHO cells, HEK cells, NIH3T3 cells and HeLa cells, and also transiently transfected cells or primary cells. Within the meaning of the invention, biological membrane bodies are also artificially produced membrane bodies in which, for example, a lipid bilayer encloses a limited volume of an aqueous medium (vesicles). These membrane bodies then preferably contain at least one biological component, e.g. a polypeptide which is embedded in the lipid bilayer, a membrane-located enzyme, an ion channel or a G protein-coupled receptor. Within the meaning of the invention, biological membrane bodies can also be bacterial cells, fungal cells or cells of other unicellular or multicellular organisms. Within the meaning of the invention, biological membrane bodies are also, for example, fungal cell or plant cell protoplasts which are obtained by removing outer cell walls or similar structures. Within the meaning of the invention, biological membrane bodies are furthermore also membrane bodies which, like synaptosomes, for example, have been produced from the membranes of living organisms by means of cleavage or fusion, or which have been obtained by combining such preparations with synthetic lipid vesicles.

Within the meaning of the invention, “dyes” are substances which can be detected optically by detecting the electromagnetic radiation which they have emitted or which they have not absorbed.

Within the meaning of the invention, “voltage-sensitive indicators” are substances which, in dependence on an applied electrical potential difference or on the electrical potential which is present, alter their physical, optical or catalytic properties in such a way that the latter elicit a detectable signal.

The skilled person is familiar with voltage-sensitive indicators such as DIBAC [14].

Within the meaning of the invention, “pH-sensitive indicators” are substances which, in dependence on the pH, alter their physical, optical or catalytic properties in such a way that they elicit a detectable signal. A large number of such indicator dyes, for example phenol red, bromthymol blue, bromphenol blue and a lot mor that are known to the skilled person in the art.

Within the meaning of the invention, “calcium-sensitive indicators” are substances which, in the presence of calcium, alter their physical, optical or catalytic properties in such a way that they elicit a detectable signal. Examples of calcium-sensitive indicators which are known to the skilled person are aequorin and other calcium-sensitive dyes such as FURA-2 (1-[6-Amino-2-(5-carboxy-2-oxazolyl)-5-benzofuranyloxy]-2-(2-amino-5-methylphenoxy)ethane-N,N,N′,N′-tetraacetic acid, pentapotassium salt), Quin-2 (8-Amino-2-[(2-amino-5-methylphenoxy)methyl]-6-methoxyquinoline-N,N,N′,Nα-tetraacetic acid, tetrapotassium salt), Fluo-3 (1-[2-Amino-5-(2,7-dichloro-6-hydroxy-3-oxy-9-xanthenyl)phenoxy]-2-(2-amino-5-methylphenoxy)ethane-N,N,N′,N′-tetraacetic acid pentaacetoxymethyl ester), INDO-1(1-[2-Amino-5-(6-carboxy-2-indolyl)phenoxy]-2-(2-amino-5-methylphenoxy)ethane-N,N,N′,N′-tetraacetic acid pentapotassium salt), and others known in the art.

“Supported bilayers” are membranes which, on one of their sides, are in contact with, or in the immediate vicinity of, a suitable solid, porous or gelatinous material. This thereby makes them more stable mechanically, and more resistant to stress, than unsupported membranes.

Within the meaning of the invention, “active compounds” are substances which are able to exert an effect on the activity of biological molecules. Active compounds which are preferred within the meaning of the invention are those which specifically affect the activity of individual biological molecules or of groups of biological molecules. Those active compounds are particularly preferred which affect the activity of receptors and/or ion channels. Very particularly preferred active compounds are able to cure, alleviate or prevent given disease syndromes in humans or animals.

Within the meaning of the invention, “active compound screening” is the selective search for chemical or biological substances which induce a given physiological effect in a given biological system. This effect is preferably the modulation of the activity of a target molecule or the curing, alleviation or prevention of a given disease syndrome in an organism. Active compound screening is preferably carried out in a high-throughput screening (HTS) format. In this case, a large number of chemical substances are brought into contact with a target during the screening process and the effects of the chemical substances on the target are evaluated.

The invention relates, in particular, to:
1. A device for detecting a biochemical activity using a detection reaction for the said biochemical activity, characterized in that, in the device, the said biochemical activity takes place such that it is spatially separated from the said detection reaction and the spatial separation is effected by means of at least two lipid membranes which are connected by gap junctions.
   The change which is elicited by the biochemical activity (for example a change in the concentration of a chemical substance or a change in voltage) passes through the said gap junctions from the site of the biochemical activity to the site of the detection reaction. At the latter site, the change is, where appropriate, amplified or rendered detectable by the detection reaction and finally detected. The detection itself can take place anywhere.
2. A device as described in item 1, with the said biochemical activity taking place in a membrane body.
3. A device as described in item 1, with the said biochemical activity taking place in a living cell.
   The living cells which are used can, for example, be cells which have been isolated by dissociation from living tissues (primary cultures). It is furthermore possible to use cells which are maintained in culture as established cell lines, such as CHO cells, HEK cells, NIH3T3 cells or HeLa cells, or else transiently transfected cells or primary cells.
4. A device as described in one of items 1 to 3, with the detection reaction being a reaction with a dye.
5. A device as described in one of items 1 to 3, with the detection reaction being a reaction with a calcium-sensitive indicator.
6. A device as described in one of items 1 to 3, with the detection reaction being a reaction with a voltage-sensitive indicator.
7. A device as described in one of items 1 to 3, with the detection reaction being a reaction with a pH-sensitive indicator.
8. A device as described in one of items 1 to 7, with one of the lipid membranes being in the form of a supported bilayer.
9. A device as described in item 8, with the supported bilayer being in the form of a planar supported bilayer.
10. A device as described in item 8, with the supported bilayer being in the form of a spherical supported bilayer.
11. A device as described in item 8, with the supported bilayer being applied to a spherical silica support.
12. A device as described in item 8, with the supported bilayer being located on a Transil® particle.
13. A device as described in one of items 1 to 7, with at least one lipid membrane spanning the end of a capillary. When using such a set-up, a detection reaction can take place in a glass capillary as is currently used, for example, for what are termed “patch-clamp” applications [12].
14. A device for determining biochemical activities, which device contains a multiplicity of devices as described in one of items 1 to 13.
15. A method for detecting a biochemical activity using a detection reaction, characterized in that the said biochemical activity takes place such that it is spatially separated from the said detection reaction and in that the spatial separation is effected by means of at least two lipid membranes and the lipid membranes are connected by gap junctions.
16. Method as described in item 15, with the said biochemical activity constituting a reaction to the presence of an active compound.
17. Use of a device as described in one of items 1 to 13 or of a device as described in item 14 for active compound screening.

### BRIEF DESCRIPTION OF THE FIGURES

FIG. 1

The figure shows a set-up according to the invention for determining biological activity, with a target molecule (simultaneously the site of the biochemical activity) (1), the site, which is spatially separated therefrom, of the detection reaction (2), a first (3) and a second (4) lipid membrane, connexins (5) and gap junctions (6). The site of the biochemical reaction (1) is spatially separated from the site of the detection reaction (2) but nevertheless functionally connected to it by way of gap junctions (6). The first lipid membrane (3) has a planar form.

FIG. 2

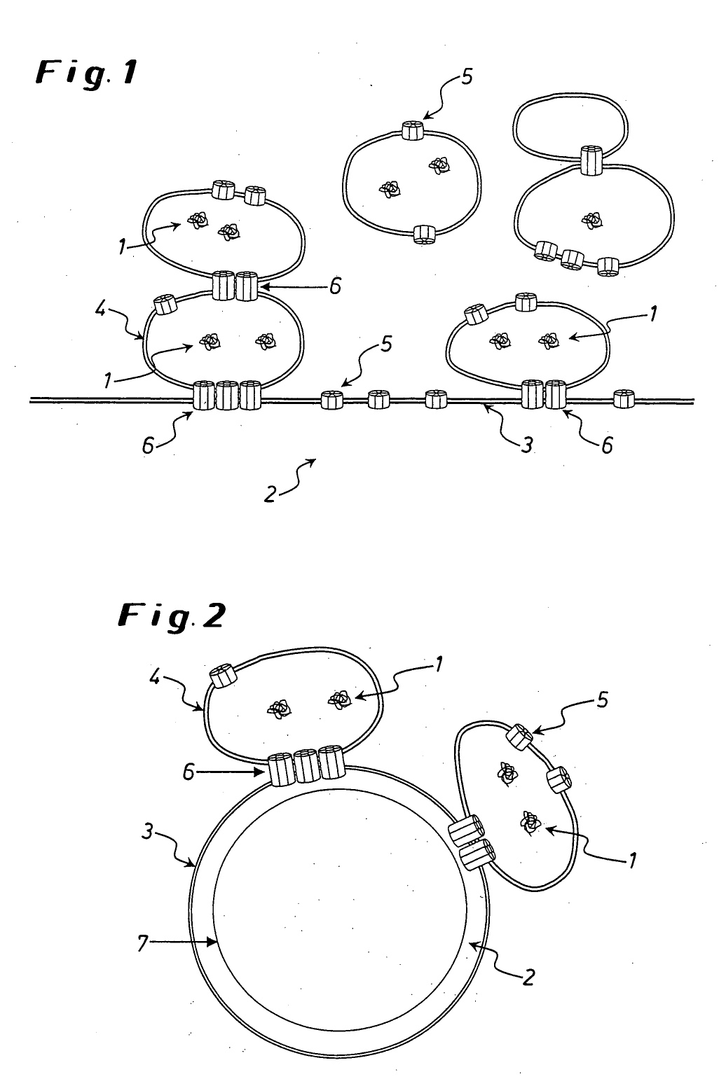

The figure shows a set-up according to the invention for determining biological activity, with a target molecule (simultaneously the site of the biochemical activity) (1), the site, which is spatially separated therefrom, of the detection reaction (2), a first (3) and a second (4) lipid membrane, connexins (5) and gap junctions (6). The detection reaction takes place below the lipid membrane (3) on a particle (e.g. on a Transil® particle) which possesses a core (7) and a lipid membrane (3). Two membrane bodies which are coupled to the particle by way of gap junctions are depicted.

The foregoing is only a description of a nonlimiting number of embodiments of the present invention. It is intended that the scope of the present invention extend to the full scope of the appended issued claims and their equivalents.

### EXAMPLES

### Example 1

### Supported Bilayer Membranes Containing Inserted Connexins

Connexins are purified from the liver and renatured, as described in the literature [4]. The NIMBUS method is used to insert the connexins into a bilayer membrane which is supported on Transil® particles. The method can naturally be carried out not only with connexin Cx32 from the liver but also, in an analogous manner, with other connexins, such as connexin Cx43 from heart muscle and virtually all other connexins as well as with proteins of comparable structure from other organisms, such as innexins from invertebrates or plasmodesmata from plants.

In many cases, it is important to be able to adjust the membrane potential. It is therefore expedient to use the same method to additionally insert a potassium-selective ion channel into the membrane, with this channel then making it possible to use the external potassium concentration to make a rough adjustment of the membrane potential of the Transil® particle. Transil® particles which have been modified in this way are described below as being “connexon-potassium channel particles”.

### Example 2

### Coupling Transil® Particles to Hepatocytes

The above-described connexon-potassium channel particles are brought into contact with dissociated hepatocytes. The skilled person is familiar with methods for isolating dissociated hepatocytes. Since hepatocytes also contain connexin Cx32, they become attached to the particles and form gap junctions with them. The gap junctions open and form a conductive connection between the hepatocytes and particles. This is demonstrated by a dye, e.g. Lucifer yellow [13], diffusing into the cells when the particles have previously been loaded with this dye.

### Example 3

### Determining the Activity of a Nicotinergic Acetylcholine Receptor

HEK cells are transfected with cDNA for expressing connexin 32 and simultaneously or subsequently transfected with cDNA for expressing a ligand-controlled ion channel, i.e. a nicotinergic acetylcholine receptor. The methods for preparing such cells and for functionally expressing the ion channel are well known and form part of the knowledge of the average skilled person.

Cells which are expressing both proteins are now brought into contact with the particles from Example 1. They become attached to the particles and form gap junctions with them since both the particles and the cells possess connexin 32.

When a suitable agonist, such as nicotine, is used to activate the nicotinergic acetylcholine receptor, this then results in a change in the membrane potential. This change in potential is transferred, by way of the gap junctions, to the membrane of the particle and is detected optically. In order to be able to do this, the particles are loaded beforehand with a voltage-sensitive membrane-soluble dye (e.g. DIBAC [14]).

### Example 4

### Second Messenger-Coupled Receptors

Cells which possess a second messenger-coupled receptor are also transfected with a connexin such that they form this connexin in their membranes in addition to the membrane proteins which are present in the native state. The cells are then brought into contact with particles from Example 1 which have been prepared using a connexin which is compatible with the transfected connexin. The cells become attached to the particles. A connection between the cells and the interior of the particles is established by means of the gap junctions. If the second messenger-coupled receptors are now activated in the cells, the intracellular reaction products then also diffuse into the particles and can be detected in these particles using an appropriate chemical reaction. This reaction could, for example, be that of using FURA-2 to detect an increase in calcium concentration, with this detection method having been widely documented in detail in the literature.

### Example 5

### Experiments Performed on Dissociated Heart Cells

In the native state, dissociated heart cells contain connexin Cx43 [15]. They are therefore able to attach to particles from Example 1 which have been prepared using connexin Cx43. If the cells are now treated with active compounds which activate receptors which are present in the heart cells, the effects can then be detected, for example, using the methods described in Examples 3 and 4.

References

[1] Austin, C. D. (1993) The Connexins: A Family of Gap Junction Proteins, Einstein Quarterly Journal of Biology and Medicine 10, 133-142
[2] Goodenough, D. A., J. A. Goliger, and D. L. Paul (1996) Connexins, Connexons, and intercellular communication Annu. Rev. Biochem. 65:475-502
[3] Jordan K., Solan J. L., Dominguez M., Sia M., Hand A., Lampe P. and Laird D. W. (1999) Trafficking, Assembly, and Function of a Connexin43-Green Fluorescent Protein Chimera in Live Mammalian Cells. Molecular Biology of the Cell 10, 2033-3050
[4] Mazet et al. (1992) Voltage Dependence of liver gap-junction channels reconstituted into liposomes and incorporated into planar bilayers. European Journal of Biochemistry 210, 249-256
[5] Brewer (1991) Reconstitution of lens channels between two membranes. Chapter 19 in: Biophysics of Gap Junction Channels, Editor: C. Peracchia, CRC Press Boca Raton, Ann Arbor, Boston
[6] Phelan (2000) Gap Junction Communication in Invertebrates: The Innexin Gene Family, Current Topics in Membranes 49, 389-422
[7] Knight et al., A functional assay for G-protein-coupled receptors using stably transformed insect tissue culture cell lines. Anal Biochem. 2003. 320(1):88-103
[8] Hanke, W. (1985) Reconstitution of Ion Channels. CRC Critical Reviews Biochemistry 19, 1-44
[9] Sackmann E. and Tanaka M. (2000) Supported Membranes on soft polymer cushions: fabrication, characterization and applications TIBTECH 18, 58-64
[10] Loidl-Stahlhofen et al. (2001) Solid-Supported Biomolecules on Modified Silica Surfaces—A Tool for Fast Physicochemical Characterization and High-Throughput Screening, Advanced Materials 13, 1829-1834
[11] Raguse et al. (1998). Tethered Lipid Bilayer Membranes: Formation, and Ionic Reservoir Characterization, Langmuir 14, 648-659
[12] Hamill, O. P., A. Marty, E. Neher, B. Sakmann and F. J. Sigworth (1981). Improved patch-clamp techniques for high-resolution current recording from cells and cell-free membrane patches. Pflügers Archiv 391, 85-100
[13] Cao et al. 1998. Journal Cell. Sci., 111, 31-43
[14] Whiteaker et al. 2001. Journal of Biomolecular Screening 6, 305-312
[15] Yeager et al. 1998. Current Opinion Struct. Biol. 8, 517-524

1. A device for spatially separating a biochemical activity from a detection reaction that detects the biochemical activity, the device comprising (a) a first membrane body comprising the biochemical activity, the first membrane body further comprising at least one lipid membrane, and (b) a second membrane body comprising a detection reaction for detecting the biochemical activity, the second membrane body further comprising at least one lipid membrane, and wherein the first and second membrane bodies are connected by gap junctions.

2. The device according to claim 1, wherein the biochemical activity occurs in a synthetic membrane body.

3. The device according to claim 1, wherein the biochemical activity occurs in a living cell.

4. The device according to claim 1, wherein the detection reaction comprises a dye.

5. The device according to claim 1, wherein the detection reaction comprises a calcium-sensitive indicator.

6. The device according to claim 1, wherein the detection reaction comprises a voltage-sensitive indicator.

7. The device according to claim 1, wherein the detection reaction comprises a pH-sensitive indicator.

8. The device according to claim 1, wherein one of the lipid membranes is in the form of a supported bilayer.

9. The device according to claim 8, wherein the supported bilayer is a planar supported bilayer.

10. The device according to claim 8, wherein the supported-bilayer is a spherical supported bilayer.

11. The device according to claim 8, wherein the supported bilayer is applied to a spherical silica support.

12. The device according to claim 8, wherein the supported bilayer comprises a silica support to which a lecithin lipid bilayer has been applied.

13. The device according to claim 1, wherein at least one lipid membrane spans an end of a capillary.

14. A device for determining biochemical activities, wherein the device comprises a multiplicity of the devices according to claim 1.

15. A method for detecting a biochemical activity, the method comprising:
a) providing the device of claim 1;
b) effecting the biochemical activity in the first membrane body of the device so that the biochemical activity is spatially separated from the detection reaction; and
c) observing the detection reaction in the second membrane body.

16. The method according to claim 15, wherein the biochemical activity comprises a reaction induced by the presence of an active compound.

17. A method for screening active compound, the method comprising:
a) providing the device of claim 1;
b) contacting one or more compounds with the first membrane body of the device under conditions suitable for performing the biochemical activity; and
c) determining the detection reaction in the second membrane body.
